(19) United States
(12) Patent Application Publication
(10) Pub. No.: US 2003/0104480 A1
(43) Pub. Date: Jun. 5, 2003

(54) Prion capture

(75) Inventors: Kailing Wang, Carlingford (AU); Chenicheri Hariharan Nair, Old Greenwich, CT (US); Mina Obradovic, Mosman (AU)

Correspondence Address: BAKER & MCKENZIE, 805 THIRD AVENUE, NEW YORK, NY, 10022, US

(73) Assignee: Gradipore Limited, Frenchs Forest NSW (AU)

(21) Appl. No.: 10/233,788

(22) Filed: Sep. 3, 2002

(30) Foreign Application Priority Data
Aug. 31, 2001 (AU) PR7409

(51) Int. Cl. G01N 33/53; C07K 14/47

(52) U.S. Cl. 435/7.1; 530/350

(57) ABSTRACT

The claims relate to methods and assays for capturing or detecting prions in a sample using prion-binding materials as well as methods of separating prions from a sample. A method selects a prion-binding material in the form of fibrin(ogen), fibrin(ogen)-related material, fibrin(ogen)-derived material, or mixtures thereof, and contacts a sample with the prion-binding material, such that prions contained in the sample are bound to or associated with the prion-binding material. In another aspect, an assay for detecting the presence of prions obtains prion-binding material in the form of fibrin(ogen), a fibrin(ogen)-related material, a fibrin(ogen)-derived material, or mixtures thereof, contacts the prion-binding material with the sample which may contain prions such that prions contained in the sample are bount to or associated with the prion-binding material, and tests for the presence of prions associated with or bound to the prion-binding material.

### CROSS-REFERENCE TO RELATED APPLICATIONS

The present application claims benefit of an Australian provisional application No. PR 7409 filed Aug. 31, 2001.

### FIELD

The present invention relates to methods and assays for capturing, detecting or binding prions.

### BACKGROUND

Bovine spongiform encepalopathy (BSE), scrapie of sheep, Kuru and Creutzfeldt-Jakob disease (CJD) of humans are only a few examples of a group of neurodegenerative disorders named transmissible spongiform encephalopathies (TSE) which are characterized by loss of motor control, dementia, paralysis, blindness, wasting and eventually death. These diseases may be inherited or sporadic. A risk of TSE for humans is believed to be through food products derived from BSE-infected cattle. Another transmission risk is a possible infection through human blood and blood products which originated from TSE-infected donors.

Recently, it was shown that fatal neurodegenerative diseases are caused by a newly discovered infectious pathogen named prion protein (PrP) (Prusiner, S. B., Proc. Natl. Acad. Sci. USA, 95, 13363-13383, (1998)). More precisely, the accumulation of the infectious isoform of the PrP into amyloid plaques results in the development of the disease. Different isoforms of PrP have been identified as a normal cellular form (PrP^{c}) and a highly infectious scrapie form (PrP^{Sc}). The PrP^{Sc} form of the protein was found to be protease and detergent resistant, while PrP^{c} has been shown to be sensitive to the conventional treatment processes causing protein degradation and denaturation. Although identical in amino acid sequence, the two proteins have been shown to have different conformational characteristics, with PrP^{c} containing more [symbol] -helical structure than its infectious counterpart. Nevertheless, presently there has been no effective method of discriminating between the two proteins by way of immunoreagents. Only recently, plasminogen was recognized as the first naturally occurring PrP^{Sc}-binding protein that can distinguish between PrP^{c} and PrP^{Sc} (Fischer, M. B., Roeckl, C., Parizek, P., Schwarz, H. P., Aguzzi, A. Nature, 408, 479-483, 2000).

Blood is likely to be a carrier of TSE infectivity (Houston, F, Foster, J D, Chong, A, Hunter, N and Bostock, C J. Transmission of BSE by blood transfusion in sheep. Lancet, 356, 999-1000, 2000; Brown P, Cervenakova, L, Diringer, H. Blood infectivity and prospects for a diagnostic screening test in Creutzfeldt-Jakob disease. J. Lab. Clinic. Med., 137, 5-13, 2001) and fibrin(ogen) is secreted into the circulation with normal levels of 2-3 mg/ml in plasma. (For convenience only, the terms fibrin, fibrin(ogen), and fibrinogen are used interchangeably in this application). Fibrinogen is a 340 kDa heterotrimer protein composed of disulfide bond linked A [symbol] , B [symbol] and [symbol] chains. Fibrin(ogen) has recently been found to bind to non-digested PrP27-30, but not PrP^{Sc}, however, the underlying structural basis and the pathogenetic significance for this interaction remain unknown (Fischer, et al. 2000).

At present, prions are present in very low numbers in diseased animals and detection is extremely difficult in ‘normal’ biological samples. Currently, there are no commercial non-invasive detection assays that allow accurate and reliable detection of prions in infected individuals prior to the development of clinical symptoms of disease. A few tests that are in use are able to detect prions in brain tissue and the spinal cord only and are largely used on animals and humans in their post-mortem state. For humans, the early detection of exposure to prions may assist in management to prevent or delay clinical symptoms of a prion-related disease. For example, one of the challenges of the “mad cow disease” epidemic is the provision of a sensitive and reliable diagnostic test to detect the presence of infectious prions at an earlier stage of the disease without requiring post-mortem brain samples. Although a few blood-screening test methods are in different stages of development, inadequate amounts of PrP^{Sc} present early in the disease epitomize the difficulties in overcoming the inherent sensitivity threshold of the methods.

Processes such as fractionation, chromatography and viral filtration, which are a routine part of the purification and manufacturing process of many biologicals, have been demonstrated to substantially remove spiked PrP^{Sc}. Whether or not these processes are capable of completely removing all TSE infectivity is not known, as this is dependent on the quantity of TSE infectivity actually present as well as on the accuracy of the experimental models.

### SUMMARY

The present application relates to methods and assays for capturing or detecting prions in a sample using prion-binding materials. The present application also relates to separating prions from a sample.

In one aspect, a method for capturing prions selects a prion-binding material in the form of fibrin(ogen), fibrin(ogen)-related material, fibrin(ogen)-derived material, or mixtures thereof, and contacts a sample with the prion-binding material so as to bind the prions with the prion-binding material.

In another aspect, an assay for detecting the presence of prions in an animal obtains prion-binding material in the form of fibrin(ogen), a fibrin(ogen)-related material, a fibrin(ogen)-derived material, or mixtures thereof, contacts the prion-binding material with a sample which may contain prions such that prions contained in the sample are bound to or associated with the prion-binding material, and tests for the presence of prions associated with or bound to the prion-binding material.

In another aspect, a method separates prions from a sample by contacting a sample containing prions with prion-binding material in the form of fibrin(ogen), a fibrin(ogen)-related material, a fibrin(ogen)-derived material, or mixtures thereof, so as to bind the prions with the prion-binding material, and removes the prion-binding material from the sample.

These and other features of the claims will be appreciated from review of the following detailed description of the application along with the accompanying figures.

### BRIEF DESCRIPTION OF THE DRAWINGS

FIG. 1 is a block diagram of a method for capturing prions;

FIG. 2 is a block diagram of an assay for detecting prions;

FIG. 3 shows Western blots of SDS-PAGE analyses of the separation of PrP^{c} from fibrinogen using a 800 kDa separation membrane in a membrane-based electrophoresis cassette;

FIG. 4 shows Western blots of SDS-PAGE analyses of the separation of PrP^{c} from fibrinogen using a 1000 kDa separation membrane in a membrane-based electrophoresis cartridge;

FIG. 5 shows Western blots of SDS-PAGE analyses of the separation of PrP^{c} from fibrinogen using a 1500 kDa separation membrane in a membrane-based electrophoresis cartridge;

FIG. 6 shows results of in vitro interaction of human fibrinogen with the bovine prion protein. Lane 1: Plasma alone; Lane 2: Plasma and bovine brain homogenate mixture; Lane 3: Negative control-no antibody was added (Protein A agarose); Lane 4: Negative control-no antibody was added (Protein G agarose); Lane 5: Anti-PrP treated sample (Protein A agarose); Lane 6: Anti-PrP treated sample (Protein G agarose); Lane 7: Positive control (Protein A agarose); Lane 8: Positive control (Protein G agarose);

FIG. 7 shows results of in vitro interaction of human fibrinogen with the bovine prion protein. Lane 1: Fibrinogen only; Lane 2: Bovine brain homogenate+fibrinogen; Lane 3: Anti-PrP treated sample (protein A agarose); lane 4: Anti-PrP treated sample (protein G agarose); Lane 5: Negative control-no antibody was added (protein A agarose); Lane 6: Negative control-no antibody was added (protein G agarose);

FIG. 8 shows the results of in vitro interaction of human fibrinogen with the prion protein. Lane 1: Plasma only (fibrinogen); Lane 2: Bovine brain homogenate+plasma (fibrinogen); Lane 3: Anti-PrP treated sample (protein A agarose); Lane 4: Anti-PrP treated sample (protein G agarose); Lane 5: Negative control-no antibody was added (protein A agarose); Lane 6: Negative control-no antibody was added (protein G agarose); Lane 7: Anti-fibrinogen treated sample (protein A agarose); Lane 8: Anti-fibrinogen treated sample (protein G agarose);

FIG. 9 shows results of in vitro interaction of fibrinogen with the prion protein. Lane 1: Plasma only (fibrinogen); lane 2: Bovine brain homogenate+plasma (fibrinogen); Lane 3: Negative control-no antibody was added (protein A agarose); Lane 4: Negative control-no antibody was added (protein G agarose); Lane 5: Anti-PrP treated sample (protein A agarose); Lane 6: Anti-PrP treated sample (protein G agarose); Lane 7: Anti-fibrinogen treated sample (protein A agarose); Lane 8: Anti-fibrinogen treated sample (protein G agarose); and

FIG. 10 shows the results of a human fibrinogen-coated ELISA-like assay.

### DETAILED DESCRIPTION

Embodiments for capturing or detecting prions according to the present claims are described in non-limiting detail below. These embodiments may also be used to separate prions from a sample.

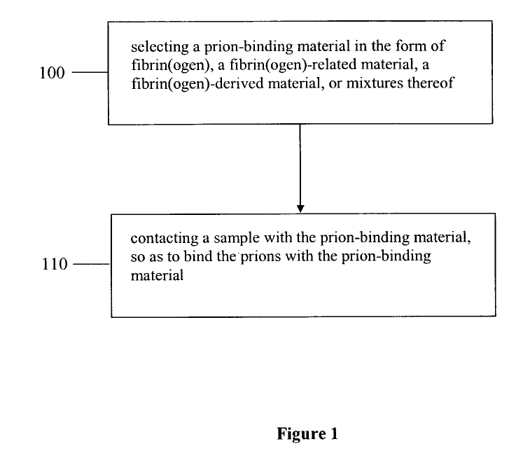

FIG. 1 refers to a block diagram of a method for capturing prions in accordance with one aspect of the present claims. Block 100 depicts selecting a prion-binding material in the form of fibrin(ogen), fibrin(ogen)-related material, fibrin(ogen)-derived material, or mixtures thereof. For example, suitable prion-binding materials may be fibrinogen degradation products, fibrinogen derivatives, fibrin, soluble fibrin, fibrin degradation products, fibrin derivatives, fibrin cross-link derivatives or mixtures or combinations thereof. In one embodiment, fibrin(ogen) is the prion-binding material. However, other prion-binding materials known in the art may also be used.

The prion-binding material may be used with or without a support matrix. In one embodiment, the prion-binding material may be immobilized onto a suitable support matrix. For example, the support matrix may be magnetic beads, membranes, resins, filters or plates, or any other suitable support matrices known in the art. In another embodiment the prior-binding material is used in isolation without a support matrix.

Referring back to FIG. 1, block 110 depicts contacting a sample with the prion-binding material, so as to bind prions with the prion-binding material. In one embodiment, the sample contains normal prions. In another embodiment, the sample contains infectious prions. In another embodiment, the sample contains a combination of normal and infectious prions. In one embodiment, prion-binding material is provided in a housing or column and a sample is passed through or by the prion-binding material. The selection of a suitable housing or column is readily ascertainable by the skilled practitioner. After passing through or by the prion-binding material, the sample can be collected for subsequent use. In another embodiment, the sample is contacted with prion-binding material in isolation without a support matrix or column. In this manner, one embodiment obtains a sample substantially free from prions.

Suitable samples may be biological materials obtained from animals or humans. For example, blood, plasma, serum, blood cell products or extracts, cerebrospinal fluid (CSF), tissue homogenates, urine, semen or combinations thereof may serve as suitable samples. However, other samples containing prions are known in the art and may also be used.

In another embodiment, the prion-binding material containing prions may be collected. The collected prion-bound material may be further treated or processed to obtain prions. For example, the prions may be removed from the prion-binding material by enzymatic digestion, physical treatment or chemical treatment. The physical treatment can include sonication, agitation or centrifugation, for example. However, other methods of removing prions from prion-binding material are readily ascertainable by one skilled in the art and may also be used. Alternatively, the material may be treated or assayed to detect the presence of prions in situ.

In one embodiment, prions are separated from a sample by contacting a sample containing prions with prion-binding material in the form of fibrin(ogen), a fibrin(ogen)-related material, a fibrin(ogen)-derived material, or mixtures thereof, such that prions contained in the sample are bound to or associated with the prion-binding material, and then removing the prion-binding material from the sample.

In one example, prions are separated from a sample by contacting the sample containing prions with prion-binding material such that prions contained in the sample are bound to or associated with the prion-binding material, placing the sample containing the prions bound to the prion-binding material in a first interstitial volume of an electrophoresis apparatus comprising a separation membrane having a defined pore size, a first restriction membrane disposed between a first electrode zone and the separation membrane so as to define a first interstitial volume therebetween, and a second restriction membrane disposed between a second electrode zone and the separation membrane so as to define a second interstitial volume therebetween, applying an electric potential between the first and second interstitial volumes whereby at least some components in the sample other than the prions bound to the prion-binding material are caused to move out of the first interstitial volume through the separation membrane or a restriction membrane while the bound prions in the sample are substantially retained in the first interstitial volume, and maintaining this step until the desired amount of components are removed from the sample to form a sample of separated prions. In another example, the prion-bound material may be caused to move through the separation membrane to the second interstitial volume while other contaminating components in the sample remain in the first interstitial volume. In one example, the pore size of the separation membrane is below 1000 kDa so that the prion-bound material cannot pass there through. In another example, the pore size of the separation membrane is above 1000 kDa so that the prion-bound material can pass there through. In one example, the pore size of the separation membrane is below 1000 kDa. In another example, the pH of the sample is between pH 4.6-9.0. However, practitioners in the art understand that other pH's and other pore sizes may be used.

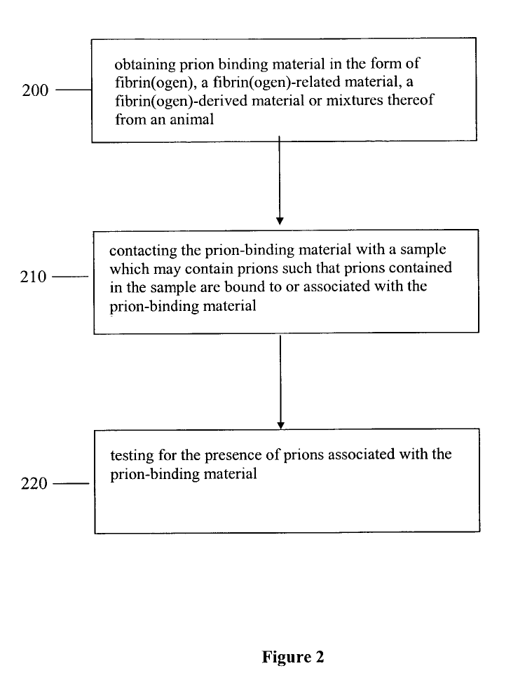

FIG. 2 refers to a block diagram of an assay for detecting the presence of prions in accordance with one aspect of the present claims. The prions may be derived from any animal that contains prions or prion-binding material. For example, the animal may be human, a live-stock animal, a cow, pig, sheep, rabbit, mouse, or any other animal known in the art that contains prions or prion-binding material.

Block 200 depicts obtaining prion-binding material in the form of fibrin(ogen), fibrin(ogen)-related material, fibrin(ogen)-derived material or mixtures thereof from an animal. For example, in one embodiment, fibrin(ogen) is obtained from an animal. The animal may be alive or dead when obtaining the prion-binding material from the animal.

Block 210 depicts contacting the prion-binding material with a sample that may contain prions such that prions contained in the sample are bound to or associated with the prion-binding material.

Block 220 depicts testing for the presence of prions bound to or associated with the prion-binding material. In one embodiment, testing for the presence of prions occurs in animal carcasses prior to use for human or animal consumption. Alternatively, the animal may be alive and testing determines whether the animal is harboring prions or has been exposed to prions. Block 220 tests for the presence of normal prions, infectious prions, or combinations thereof. In one embodiment, the assay differentiates infectious prions from normal prions.

In one embodiment, the assay mixes a sample with prion-binding material in the form of fibrin(ogen), fibrin(ogen)-related material, fibrin(ogen)-derived material or mixtures thereof, and detects a change in the prion-binding material when prions are bound to or associated with the prion-binding material. The prion-binding material can be used in isolation without a support matrix or immobilized to any suitable support matrix. For example, magnetic beads, membranes, resins, filters, and plates may be used. Moreover, other suitable support matrices are readily ascertainable by one skilled in the art and may also be used.

In one example, a sample is mixed with prion-binding material in the form of fibrin(ogen). In this example, fibrin(ogen) forms aggregates in the presence of prions and thus the formation of fibrin(ogen) aggregates indicates the presence of prion bound to or associated with the prion-binding material. The aggregates may be detected by any suitable means known in the art. For example, detecting an increase in molecular mass of fibrin(ogen), a change in refractive index, or the formation of soluble fibrin may be used to detect a change in the prion-binding material. Of course, other methods of detecting fibrinogen aggregates known in the art may also be used.

In one embodiment, the assay may be automated. In another embodiment, samples are tested as a screening method for the presence of prions. The prions may be normal prions, infectious prions, or a combination of both normal and infectious prions.

Fibrin(ogen) is one example of a prion-binding material that interacts specifically with prion protein with high efficiency. The use of fibrin(ogen) or related compounds results in rapid concentration and enrichment of prion proteins in a prion diagnostic assay system for screening prions in blood fractions, plasma, biological fluids, or other media containing prions. In one embodiment, fibrin(ogen) or other prion-binding materials are immobilized to matrices and used as prion clearance devices. Any binding differences between infectious and non-infectious prions may be utilized to carry out differential binding of prion populations.

As prions have been found to associate with fibrinogen in vitro, prions have a similar association with fibrinogen in vivo. As such, an assay according to the claims is capable of detecting prions by analyzing fibrinogen from animals suspected of being infected or carrying prions. Isolating fibrinogen containing bound prions from blood, for example, allows the detection of prions, as they are present in higher concentrations than in other samples. In one example, an assay according to the present claims obtains blood from an animal or human patient, separates the plasma, and isolates the fibrinogen. The isolated fibrinogen is then tested for the presence of bound prions.

Fibrin(ogen) is also suitable for the concentration and detection of prion protein, and also useful for improving prion clearance from prion-contaminated biologicals or other samples. In one embodiment, fibrin(ogen) or related equivalents permit rapid concentration and enrichment of prion proteins in a prion diagnostic assay system for screening prions in blood fractions, plasma or other biological fluids.

In one embodiment, fibrin(ogen) or related compounds is used as an indicator for prion surrogate detection in blood. For example, by separating fibrin(ogen) from plasma, prions present in blood or in plasma are captured and fibrin(ogen)-bound prion is subsequently detected by Western Blot or ELISA. This approach eliminates the masking effect of non-specific proteins and thus increases the sensitivities for both assays. In other embodiments, fibrin(ogen) may be immobilized on magnetic beads or other support matrices known in the art. In one embodiment, after immobilized fibrin(ogen) material is subjected to plasma pools, biologicals/biopharmaceuticals or any potentially prion-contaminated materials, prion clearance may be achieved by retrieving these beads from starting material or by filtering contaminated material through the fibrin(ogen)-immobilized matrices.

To assist in understanding the present application, the following examples are included and describe the results of a series of experiments. The following examples relating to this application should not be construed to specifically limit the application or such variations of the application, now known or later developed, which fall within the scope of the application as described and claimed herein.

### Analytical Methods and Reagents

Prions

Bovine prion protein was sourced from bovine brain homogenate, homogenized in PBS (Phosphate buffered saline)/Triton X-100 (0.5% v/v) containing Cocktail protease inhibitor tablets (1 tablet/50 ml) (1836145, Roche, Germany). Homogenized brain was centrifuged (3,000×g, 10 minutes, 4° C.). The supernatant collected was solubilized for 30 minutes on ice and subsequently centrifuged (15,000×g, 2 hours, 4° C.). All supernatant was collected and pooled together and stored at −80° C. until ready to use.

The present experiments were carried out with non-infectious prions due to the difficulty in handling infectious prions and the lack of infectious bovine prions in Australia. However, due to the same amino acid make up and similar characteristics between infectious and non-infectious prions, infectious prions also react with fibrin(ogen) and related compounds in an analogous manner.

Fibrinogen

Human fibrinogen was either obtained from normal human plasma obtained from the Australian Red Cross or pure human fibrinogen (F-3879, Sigma, USA)

Membrane-Based Electrophoresis

A number of membrane-based electrophoresis apparatus developed by Gradipore Limited, Australia were used in the following experiments. In summary, the apparatus typically included a cartridge which housed a number of membranes forming two chambers, cathode and anode connected to a suitable power supply, reservoirs for samples, buffers and electrolytes, pumps for passing samples, buffers and electrolytes, and cooling means to maintain samples, buffers and electrolytes at a required temperature during electrophoresis.

The cartridge contained three substantially planar membranes positioned and spaced relative to each other to form two chambers through which sample or solvent can be passed. A separation membrane was positioned between two outer membranes (termed restriction membranes as their molecular mass cut-offs are usually smaller than the cut off of the separation membrane). When the cartridge was installed in the apparatus, the restriction membranes were located adjacent to an electrode. The cartridge is described in AU 738361, which description is incorporated herein by reference.

Description of other suitable membrane-based electrophoresis apparatus can be found in U.S. Pat. No. 5,039,386 and U.S. Pat. No. 5,650,055 and is incorporated herein by reference. However, other membrane-based electrophoresis may also be used.

Polyacrylamide Gel Electrophoresis (PAGE)

Standard PAGE methods were employed as set out below.

Reagents: 10×SDS Glycine running buffer (Gradipore Limited, Australia), dilute using Milli-Q water to 1× for use; 1×SDS Glycine running buffer (29 g Trizma base, 144 g Glycine, 10 g SDS, make up in RO water to 1.0 L); 10×TBE II running buffer (Gradipore), dilute using Milli-Q water to 1× for use; 1×TBE II running buffer (10.8 g Trizma base, 5.5 g Boric acid, 0.75 g EDTA, make up in RO water to 1.0 L); 2×SDS sample buffer (4.0 ml, 10% (w/v) SDS electrophoresis grade, 2.0 ml Glycerol, 1.0 ml 0.1% (w/v) Bromophenol blue, 2.5 ml 0.5M Tris-HCl, pH 6.8, make up in RO water up to 10 ml); 2×Native sample buffer (10% (v/v) 10×TBE II, 20% (v/v)PEG 200, 0.1 g/L Xylene cyanole, 0.1 g/L Bromophenol blue, make up in RO water to 100%); Coomassie blue stain (Gradipure™, Gradipore Limited). Note: contains methanol 6% Acetic Acid solution for de-stain.

Molecular weight markers (Recommended to store at −20° C.): SDS PAGE (e.g. Sigma wide range); Western Blotting (e.g. color/rainbow markers).

SDS PAGE with Non-Reduced Samples

To prepare the samples for running, 2×SDS sample buffer was added to sample at a 1:1 ratio (usually 50 [symbol] L /50 [symbol] L) in the microtiter plate wells or 1.5 ml tubes. The samples were incubated for 5 minutes at approximately 100° C. Gel cassettes were clipped onto the gel support with wells facing in, and placed in the tank. If only running one gel on a support, a blank cassette or plastic plate was clipped onto the other side of the support.

Sufficient 1×SDS glycine running buffer was poured into the inner tank of the gel support to cover the sample wells. The outer tank was filled to a level approximately midway up the gel cassette. Using a transfer pipette, the sample wells were rinsed with the running buffer to remove air bubbles and to displace any storage buffer and residual polyacrylamide.

Wells were loaded with a minimum of 5 [symbol] l of marker and the prepared samples (maximum of 40 [symbol] l). After placing the lid on the tank and connecting leads to the power supply the gel was run at 150V for 90 minutes. The gels were removed from the tank as soon as possible after the completion of running, before staining or using for another procedure (e.g. Western blot).

Staining and De-Staining of Gels

The gel cassette was opened to remove the gel which was placed into a container or sealable plastic bag. The gel was thoroughly rinsed with tap water, and drained from the container. Coomassie blue stain (approximately 100 ml Gradipure™, Gradipore Limited, Australia)) was added and the container or bag sealed. Major bands were visible in 10 minutes but for maximum intensity, stained overnight. To de-stain the gel, the stain was drained off from the container.

The container and gel were rinsed with tap water to remove residual stain. 6% acetic acid (approximately 100 ml) was poured into the container and sealed. The de-stain was left for as long as it takes to achieve the desired level of de-staining (usually 12 hours). Once at the desired level, the acetic acid was drained and the gel rinsed with tap water.

A time course of the starting material and final product were run on 4-20% SDS-PAGE igels™ (Gradipore Limited, Australia). The gels were then stained using Gradipure™ Coomassie blue stain (Gradipore Limited, Australia) and de-stained with 6% acetic acid.

Western Blot

Following an SDS-PAGE run, the gel, nitrocellulose membrane and filter pads were all equilibrated in Schaefer Nielson Transfer Buffer (14.5 g Tris, 7.54 g Glycine, 20% (v/v) methanol in 2 l H₂O) for 30 minutes. Protein transfer from the gel onto the nitrocellulose membrane was performed using a semi dry blotting apparatus (1 hour, 15 Volts). This was followed by a blocking step whereby the membrane was incubated in 5% (w/v) skim milk made up in PBS/Tween 20 (0.05%) (30 minutes, 37° C.). When performing an anti-PrP Western Blot, the membrane was firstly incubated with anti-PrP primary antibody (RO29 Prionics, Switzerland) at 1:500 dilution in 1% (w/v) skim milk for 1 hour at room temperature. When performing an anti-Fibrinogen Western Blot, the membrane was firstly incubated with anti-fibrinogen primary antibody (A0080, DAKO, Denmark) at 1:2000 dilution in 1% (w/v) skim milk for 1 hour at room temperature. When performing either of the Western Blots the secondary antibody used was goat anti-rabbit HRP (P0448, DAKO, Denmark) at 1:1000 dilution in 1% (w/v) skim milk for 1 hour at room temperature. Following each antibody incubation, a washing step in PBS/Tween (0.05%) (3×10 minutes) was performed. The membrane was finally incubated in enhanced chemiluminescence (ECL) reagents (RPN2209, Amersham Pharmacia Biotech, UK) for approximately 1 minute. The chemiluminescent signal was detected by placing a hyperfilm (RPN1674K, Amersham Pharmacia Biotech, UK) on top of the membrane and exposing it for 2-5 minutes. Signal was visualized by developing the film using the developer and replenisher (1900943, Kodak, Australia). The film was subsequently fixed using the fixer and replenisher (1901875, Kodak, Australia).

Fibrinogen-Coated ELISA-like Assay

The fibrinogen-coated ELISA-like assay was performed according to time-resolved dissociation-enhanced fluoroimmunoassay (DELFIA) (EG&G Wallac, Turku, Finland). The supplied protocol was modified to utilize a 96-well plate which was coated with pure human fibrinogen made up in PBS (1 mg/ml) and incubated overnight at 4° C. All test sample (e.g., human platelet) dilutions were made up in DELFIA assay buffer. The fibrinogen-coated plate was washed using DELFIA Wash buffer (2×300 [symbol] l per well) before adding 200 [symbol] l of test sample per well onto the plate. As such, the plate was incubated for 1 hour at room temperature with agitation. Following incubation with the test samples, the plate was washed with DELFIA Wash buffer (4×200 ul per well). Subsequent PrP-antibody (Eu-3F4, supplied in the kit) incubation was performed for 1 hour at room temperature. Following the antibody incubation, plate was washed with DELFIA Wash buffer (6×300 ul per well). DELFIA Enhancement solution was added to the wells (200 [symbol] l per well) and the plate mixed gently for 5 minutes at room temperature before measuring the fluorescence emitted using the time resolved fluorometer VICTOR2. (EG&G Wallac, Turku, Finland)

### EXAMPLES

Association of Prions with Fibrinogen

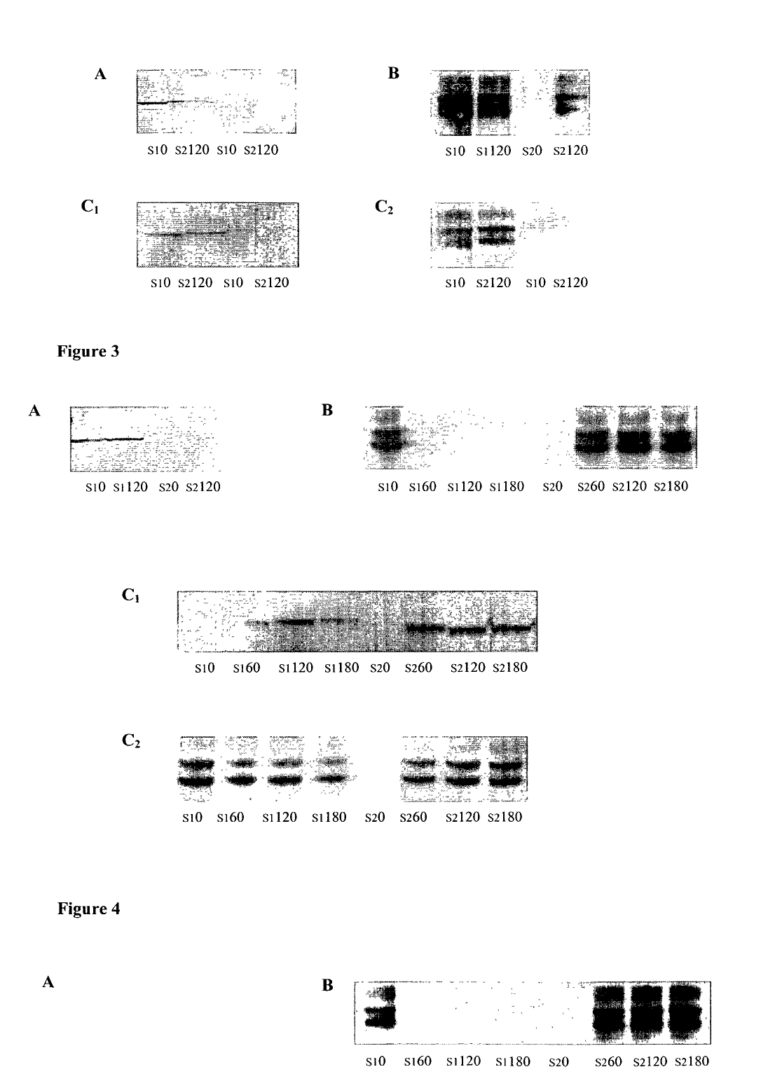

The results of the separation of PrP^{c} and fibrinogen by membrane-based electrophoresis are shown in FIGS. 3, 4 and 5. In FIG. 3, separation of PrP^{c} from human fibrinogen was carried out using a Gradiflow™ apparatus for 2 hours at 250V using a cartridge with a separation membrane of 800 kDa and two restriction membranes of 5 kDa and a Tris Borate buffer (20 mM Boric Acid, 45 mM Trizma Base, pH 9.0). Samples were taken at various intervals, separated by SDS-PAGE, blotted and then analyzed by Western blot. A) Anti-PrP Western blot of the bovine brain homogenate run; B) Anti-fibrinogen Western blot of the fibrinogen run; C₁ and C₂) Anti-PrP and anti-fibrinogen Western blot respectively of the bovine brain homogenate and fibrinogen mixture. S1 designates the sample stream and S2 designates the separation stream of the electrophoresis apparatus. The numeral after S1 or S2 relates to the time that material was sampled for analysis.

In FIG. 4, separation of PrP^{c} from human fibrinogen was carried out using the Gradiflow™ apparatus for 3 hours at 250V using a cartridge with a separation membrane of 1000 kDa and two restriction membranes of 5 kDa and a Tris Borate buffer (20 mM Boric Acid, 45 mM Trizma Base, pH 9.0). Samples were taken at various intervals, separated by SDS-PAGE, blotted and then analyzed by Western blot. A) Anti-PrP Western blot of the bovine brain homogenate run; B) Anti-fibrinogen Western blot of the fibrinogen run; C₁ and C₂) Anti-PrP and anti-fibrinogen Western blot respectively of the bovine brain homogenate and fibrinogen mixture. S1 designates the sample stream and S2 designates the separation stream of the electrophoresis apparatus. The numeral after S1 or S2 relates to the time that material was sampled for analysis.

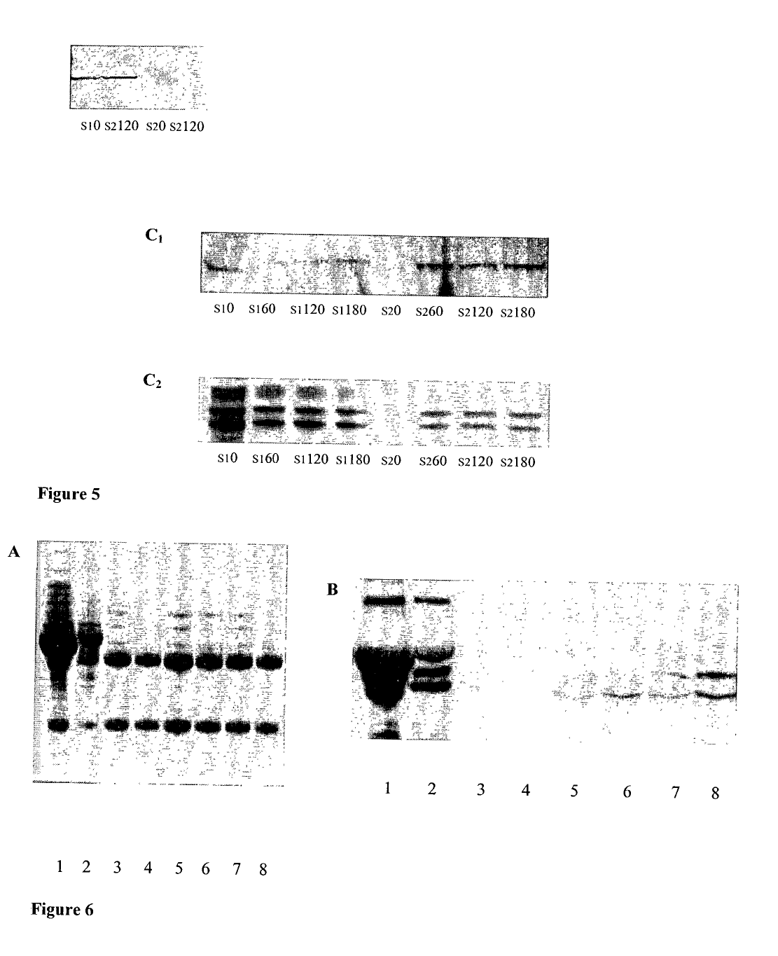

In FIG. 5, separation of PrP^{c} from human fibrinogen was carried out using the Gradiflow™ apparatus for 3 hours at 250V using a cartridge with a separation membrane of 1500 kDa and two restriction membranes of 5 kDa and a Tris Borate buffer (20 mM Boric Acid, 45 mM Trizma Base, pH 9.0). Samples were taken at various intervals, separated by SDS-PAGE, blotted and then analyzed by Western blot. A) Anti-PrP Western blot of the bovine brain homogenate run; B) Anti-fibrinogen Western blot of the fibrinogen run; C₁ and C₂) Anti-PrP and anti-fibrinogen Western blot respectively of the bovine brain homogenate and fibrinogen mixture. S1 designates the sample stream and S2 designates the separation stream of the electrophoresis apparatus. The numeral after S1 or S2 relates to the time that material was sampled for analysis.

As shown in FIGS. 3-5, human fibrinogen was transferred from the sample in the first stream (S1) chamber of the apparatus through large molecular mass cut-off separation membranes into the second stream (S2) chamber of the apparatus having a separation cartridge configuration of 5-800-5, or 5-1000-5, or 5-1500-5 (molecular mass of restriction-separation-restriction membrane configurations in a separation cartridge) when Tris/Borate buffer (20 mM Boric Acid, 45 mM Trizma Base, pH 9.0) was used. Under the same electrophoresis running conditions, PrP^{c} from bovine brain homogenate remained in the S1 chamber with starting material and did not move through the separation membrane into the S2 chamber.

When both human fibrinogen and bovine brain homogenate were mixed and the mixture treated by membrane-based electrophoresis using larger 5-1000-5 or 5-1500-5 cartridge membrane configurations, PrP^{c} was detected in the S2 chamber together with the fibrinogen. Furthermore, when the same mixture was treated by membrane-based electrophoresis using a smaller 5-800-5 cartridge membrane configuration, transfer of fibrinogen was completely blocked in the presence of PrP^{c}. Without being limited by any theory, the change in separation characteristics of fibrinogen and PrP^{c} was due to the complexing or association of PrP^{c} with fibrinogen.

By varying the electrophoresis conditions (e.g., buffer pH ranging from 4.6 to 9.0, forward or reverse polarity), the fibrinogen could not be separated from PrP^{c} by size, charge and size & charge separation modes. The movement of PrP^{c} from the sample in the S1 chamber to the S2 chamber in the electrophoresis apparatus was associated with fibrinogen, indicating a possible change in pI of PrP^{c} when fibrinogen-prion aggregates were formed. These results indicate that when fibrinogen-prion aggregates formed, the resulting complex was difficult to dissociate, even by selective electrophoretic separation conditions.

The interaction of fibrinogen with PrP^{c} was further confirmed by an immunoprecipitation experiment. Moreover, when prion samples were loaded on a 96-well plate pre-coated with fibrinogen, fibrinogen-bound prion was detected by an ELISA-like assay using anti-PrP antibody.

A mixture of bovine brain homogenate and pure fibrinogen was run through the Gradiflow™ apparatus for 3 hours at 250V using a cartridge with a separation membrane of 1500 kDa and two restriction membranes of 5 kDa at various buffer conditions. The results of all the different runs are shown in Table 1.

TABLE 1
Behaviour of PrP and fibrinogen in membrane-based electrophoresis
under different running conditions.

| Buffer Conditions/ | Fibrinogen | | Bovine PrP | |
|---|---|---|---|---|
| Polarity | Sream 1 | Stream 2 | Stream 1 | Stream 2 |
| Tris/Borate pH 9.0 | + | + | + | + |
| Forward polarity | | | | |
| HEPES/Imidazole pH 7.1 | + | + | + | + |
| Forward polarity | | | | |
| Reverse polarity | + | − | + | − |
| MES/Histidine pH 6.1 | + | − | + | − |
| Forward polarity | | | | |
| MES/Histidine pH 5.5 | + | − | + | − |
| Reverse polarity | | | | |
| GABA/Acetic Acid pH | + | − | + | − |
| 4.6 Reverse polarity | | | | |

+ present
− absent

PrP from human platelet was analyzed for interaction with human fibrinogen. A 96-well plate coated with human fibrinogen (1 mg/ml) was incubated with human platelets for 1 hour at room temperature. A subsequent washing step ensured any non-specific binding was removed. Bound PrP was detected using dissociation-enhanced fluoroimmunoassay (DELFIA) and expressed in Eu counts. Table 2 compares the binding intensity of human platelet PrP to PrP antibody and human platelet PrP to fibrinogen. The amount of PrP bound to the PrP antibody-coated plate (expressed in Eu counts) was relative to the concentration of platelet PrP used. Similarly, with an increase in platelet PrP concentration, there was an evident increase in the signal intensity (i.e. increase in Eu counts) associated with PrP binding to the fibrinogen coating. These results show a distinct trend in the PrP binding to fibrinogen reflective of PrP binding to its antibody. The results establish the interaction between human PrP and human fibrinogen.

TABLE 2
Fibrinogen/PrP interaction

| | PrP antibody coating | Fibrinogen coating |
|---|---|---|
| Human platelet | (Eu counts) | (Eu counts) |
| Neat | 671025 | 22280 |
| 1/5 dilution | 226890 | 3082 |
| 1/25 dilution | 50590 | 745 |

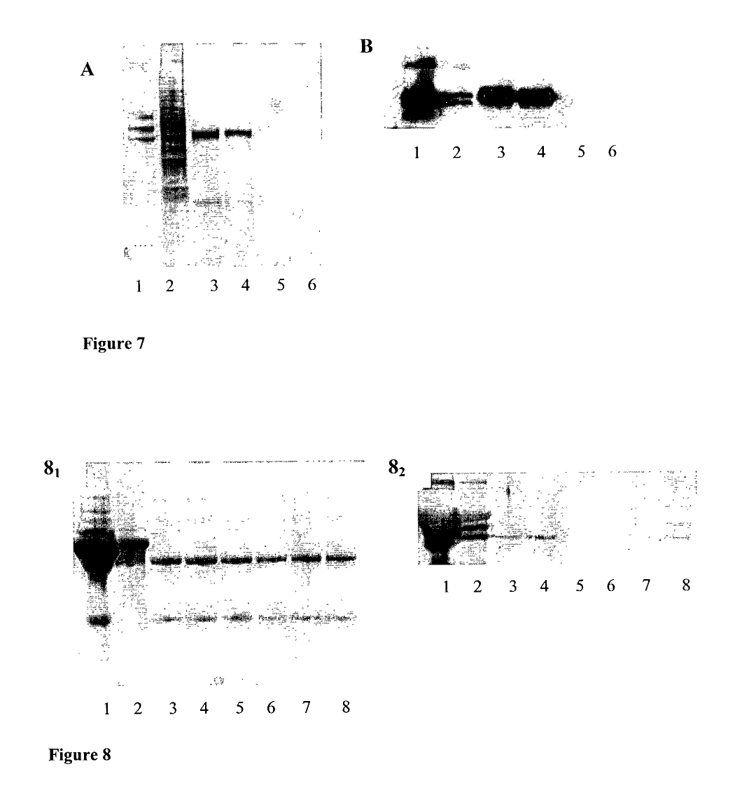

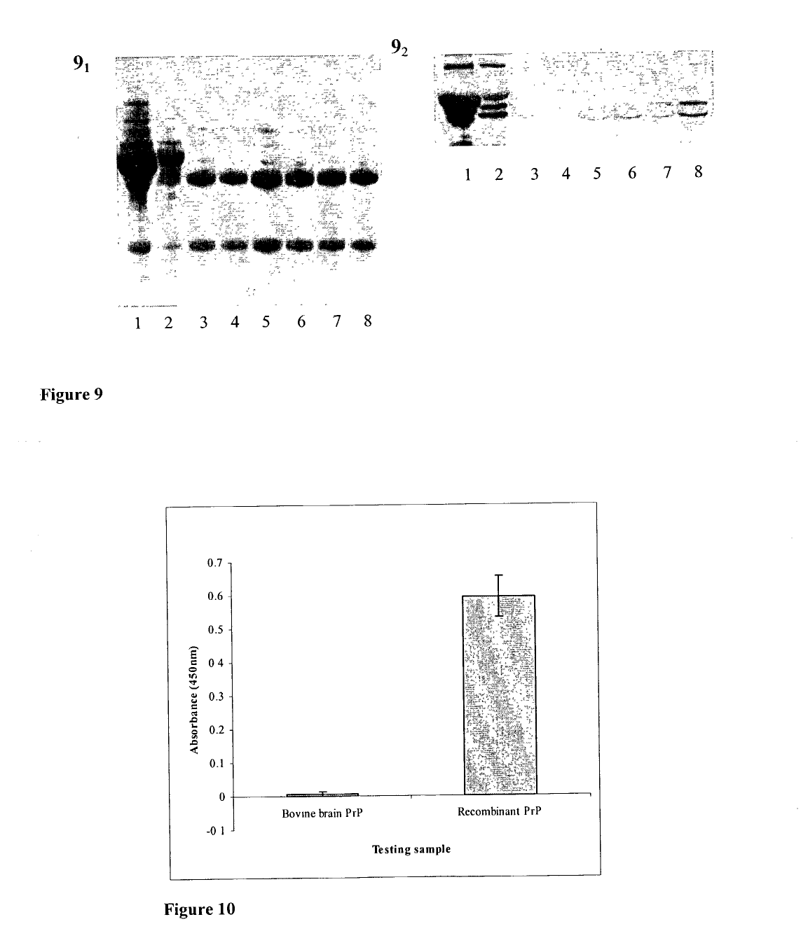

FIG. 6, FIG. 7, FIG. 8 and FIG. 9 show the results of the studies on human fibrinogen and bovine prion interaction/association. In FIG. 6, human plasma and bovine brain homogenate were mixed and incubated with anti-PrP antibody at room temperature for 2 hours to allow fibrinogen/PrP immunocomplex to be formed. This step was followed by the addition of Protein A and Protein G agarose, respectively, for a two-hour incubation at room temperature which would allow for Protein A or Protein G agarose to pull out the fibrinogen/PrP immunocomplex formed. The fibrinogen/PrP complex was then detected on a Western blot using the anti-fibrinogen antibody after SDS-PAGE separation and blotting. Panel A and B are the SDS-PAGE and the anti-fibrinogen Western Blot respectively, of the experimental samples.

In FIG. 7, pure human fibrinogen and bovine brain homogenate were mixed and incubated with anti-PrP antibody at room temperature for 2 hours to allow fibrinogen/PrP immunocomplex to be formed. This step was followed by the addition of Protein A and Protein G agarose, respectively, for a 2 hour incubation at room temperature which would allow for Protein A or Protein G to immunoprecipitate the fibrinogen-PrP immunocomplex formed. The fibrinogen/PrP complex was then detected on a Western Blot using the anti-fibrinogen antibody after SDS-PAGE separation and blotting. Panel A and B are the SDS-PAGE and the anti-fibrinogen Western Blot respectively, of the experimental samples.

In FIG. 8, human plasma and bovine brain homogenate were mixed and incubated with anti-PrP antibody at room temperature for 2 hours to allow fibrinogen/PrP immunocomplex to be formed. This was followed by the addition of Protein A and Protein G agarose, respectively for a two-hour incubation at room temperature which would allow for Protein A or Protein G agarose to pull out the fibrinogen/PrP immunocomplex formed. The fibrinogen/PrP complex was then detected on a Western blot using the anti-fibrinogen antibody. Panel 8₁ and 8₂ are the SDS-PAGE and the anti-fibrinogen Western blot respectively, of the experimental samples.

In FIG. 9, human plasma and bovine brain homogenate were mixed and incubated with anti-PrP antibody at room temperature for 2 hours to allow fibrinogen/PrP immunocomplex to be formed. This was followed by the addition of Protein A and Protein G agarose, respectively for a two-hour incubation at room temperature which would allow for Protein A or Protein G agarose to pull out the fibrinogen/PrP immunocomplex formed. The fibrinogen/PrP complex was then detected on a Western blot using the anti-fibrinogen antibody. Panel 9₁ and 9₂ are the SDS-PAGE and the anti-fibrinogen Western Blot respectively, of the experimental samples. Human prion acted similarly to bovine prion with regard to the binding to fibrinogen, thus establishing the absence of a species-related phenomenon. Accordingly, embodiments of the claims have applications in veterinary as well as human health due to the universal phenomenon regarding the association of prions with fibrinogen.

FIG. 10 shows the results of a human fibrinogen-coated ELISA-like assay. Recombinant PrP (10 ng/well) and bovine brain homogenate (containing unknown concentration of PrP), were added separately to fibrinogen-coated plate wells (1 mg/ml fibrinogen per well) and incubated for 4 hours at 37° C. The amount of PrP that bound to the fibrinogen coating was detected using an anti-PrP antibody (1/500 dilution; Prionics, Switzerland; R029), followed by a goat anti-rabbit IgG HRP conjugated antibody (1/1000 dilution; DAKO, Denmark; P0448). The color of the assay was developed using a Tolidine-based substrate and the reaction terminated using 3M HCl. The absorbance was read at room temperature, at 450 nm. The negative control sample (PBS/Tween 0.05%) was subtracted from each of the results plotted. Standard deviation was also calculated and plotted onto the graph. The results clearly show that PrP is captured and detected according to the present claims.

Gradiflow™ is a trade mark of Gradipore Limited, Australia, and is used in connection with Gradipore Limited's proprietary electrophoresis apparatus and membrane separation cartridges.

It will be appreciated by persons skilled in the art that numerous variations and/or modifications may be made to the embodiments as shown without departing from the spirit or scope of the claims as broadly described. The present embodiments are, therefore, to be considered in all respects as illustrative and not restrictive.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present claims. It is not to be taken as an admission that any or all of these matters form part of the prior art base or were common general knowledge in the field as it existed before the priority date of each claim of this application.

What is claimed is:

1. A method for capturing prions comprising:
(a) selecting a prion-binding material in the form of fibrin(ogen), a fibrin(ogen)-related material, a fibrin(ogen)-derived material, or mixtures thereof; and
(b) contacting a sample containing prions with the prion-binding material, so as to bind the prions with the prion-binding material.

2. The method according to claim 1 further comprising:
collecting the prions bound to the prion-binding material.

3. The method according to claim 2 further comprising obtaining the prions from the collected prion-binding material.

4. The method according to claim 3 wherein obtaining the prions from the prion-binding material is carried out by methods selected from the group consisting of enzymatic digestion, physical disruption, chemical treatment, and combinations thereof.

5. The method according to claim 3 whereby the physical disruption is selected from the group consisting of sonication, and centrifugation.

6. A method of separating prions from a sample, comprising;
(a) contacting a sample containing prions with prion-binding material in the form of fibrin(ogen), a fibrin(ogen)-related material, a fibrin(ogen)-derived material, or mixtures thereof, so as to bind the prions with the prion-binding material; and
(b) removing the prion-binding material from the sample.

7. The method according to claim 1 or 6 whereby the prion-binding material is selected from the group consisting of fibrinogen degradation products, fibrinogen derivatives, fibrin, soluble fibrin, fibrin degradation products, fibrin derivatives, fibrin cross-link derivatives, and combinations thereof.

8. The method according to claim 1 or 6 whereby the prion-binding material is fibrin(ogen).

9. The method according to claim 1 or 6 whereby the prion-binding material is used in isolation or immobilized to a support.

10. The method according to claim 1 or 6 whereby the prion-binding material is immobilized to a support selected from the group consisting of magnetic bead, membrane, resin, filter, column, housing, and plate.

11. The method according to claim 1 or 6 whereby the prions are normal prions.

12. The method according to claim 1 or 6 whereby the prions are infectious prions.

13. The method according to claim 1 or 6 whereby the sample is selected from the group consisting of a biological material obtained from an animal, blood, plasma, serum, cell products, cell extracts, cerebrospinal fluid (CSF), tissue homogenates, urine, semen, and combinations thereof.

14. A method for separating prions from a sample comprising:
(a) contacting the sample containing prions with prion-binding material in the form of fibrin(ogen), a fibrin(ogen)-related material, a fibrin(ogen)-derived material, or mixtures thereof, so as to bind the prions with the prion-binding material;
(b) placing the sample containing the prions bound to the prion-binding material in a first interstitial volume of an electrophoresis apparatus comprising a separation membrane having a defined pore size, a first restriction membrane disposed between a first electrode zone and the separation membrane so as to define a first interstitial volume therebetween, and a second restriction membrane disposed between a second electrode zone and the separation membrane so as to define a second interstitial volume therebetween;
(c) applying an electric potential between the first and second interstitial volumes whereby at least some components in the sample other than the prions bound to the prion-binding material move out of the first interstitial volume through the separation membrane or a restriction membrane while the bound prions in the sample are substantially retained in the first interstitial volume; and
(d) maintaining step (c) until the desired amount of components are removed from the sample to form a sample of separated prions.

15. The method according to claim 14, whereby the pore size of the separation membrane is below 1000 kDa.

16. The method according to claim 14, whereby the pH of the sample is between pH 4.6-9.0.

17. An assay for detecting the presence of prions in an animal, comprising:
(a) obtaining prion binding material in the form of fibrin(ogen), a fibrin(ogen)-related material, a fibrin(ogen)-derived material or mixtures thereof from an animal; and
(b) contacting the prion-binding material with a sample which may contain prions such that prions contained in the sample are bound to or associated with the prion-binding material;
(c) testing for the presence of prions bound to or associated with the prion-binding material.

18. The assay according to claim 17 wherein the animal is a live stock animal or a human.

19. The assay according to claim 17 wherein the live stock animal is a cow.

20. The assay according to claim 17 wherein the animal is alive when the prion-binding material is obtained.

21. The assay according to claim 17 wherein the animal is dead when the prion-binding material is obtained.

22. An assay for detecting prions comprising:
(a) mixing a sample which may contain prions with a prion-binding material in the form of fibrin(ogen), a fibrin(ogen)-related material, a fibrin(ogen)-derived material or mixtures thereof; and
(b) detecting a change in the prion-binding material when prions are bound to or associated with the prion-binding material.

23. The assay according to claim 17 or 22 wherein the prion-binding material is selected from the group consisting of fibrinogen degradation products, fibrinogen derivatives, fibrin, soluble fibrin, fibrin degradation products, fibrin derivatives, fibrin cross-link derivatives, and combinations thereof.

24. The assay according to claim 16 or 22 wherein the prion-binding material is fibrin(ogen).

25. The assay according to claim 17 or 22 wherein the prion-binding material is immobilized to a support selected from the group consisting of magnetic bead, membrane, resin, filter, column, housing, and plate.

26. The assay according to claim 17 or 22 wherein the prions are normal prions.

27. The assay according to claim 17 or 22 wherein the prions are infectious prions.

28. The assay according to claim 22 wherein the prion-binding material is fibrin(ogen) and the change in the prion-binding material is formation of aggregates of fibrin(ogen).

29. The assay according to claim 22 wherein the aggregates are detected by measurement of an increase in molecular mass of fibrin(ogen),

30. The assay according to claim 22 wherein the aggregates are detected by measurement of an increase in change in refractive index, or formation of soluble fibrin.

31. The assay according to claim 22 wherein the aggregates are detected by measurement of an increase in formation of soluble fibrin.
